(19) United States
(12) Patent Application Publication
(10) Pub. No.: US 2024/0350736 A1
(43) Pub. Date: Oct. 24, 2024

(54) Activation member and subassembly for a medicament delivery device

(71) Applicant: SHL Medical AG, Zug (CH)

(72) Inventor: Daniel Carlsson, Nacka Strand (SE)

(21) Appl. No.: 18/687,942

(22) PCT Filed: Aug. 18, 2022
(86) PCT No.: PCT/EP2022/073051

§ 371 Date: Feb. 29, 2024

(30) Foreign Application Priority Data
Sep. 1, 2021 (EP) 21194407.9

(51) Int. Cl. A61M 5/315; A61M 5/20

(52) U.S. Cl. CPC A61M 5/31501; A61M 5/2033

(57) ABSTRACT

The present disclosure provides an activation member for a medicament delivery device comprising an outer part and an inner part coaxially connected to the outer part. The outer part comprises a needle cover contact surface and the inner part comprises at least one flexible arm having an inclined end surface and a plunger rod support at its distal end. The inner part further comprises at least two spring supports configured to support a distal end of a drive spring of the medicament delivery device. The plunger rod support is configured to engage with a plunger rod of the medicament delivery device in order to inhibit a proximal movement of the plunger and the inclined end surface is configured to interact with a distal end part of the medicament delivery device, thereby disengaging the plunger rod support from the plunger rod, when the activation member is distally displaced by a needle cover of the medicament delivery device.

### TECHNICAL FIELD

The present invention relates to an activation member for a medicament delivery device which enables a two-step activation of the medicament delivery device. Further, the present invention relates to a subassembly for a medicament delivery device comprising said activation member. Further, the present invention relates to a medicament delivery device comprising said subassembly or said activation member, so that the medicament delivery device can be activated in two-steps, i.e. removing an end cap and displacing a needle cover.

### RELATED ART

Medicament delivery devices, especially disposable single-dose auto-injectors, are often intended to be used by users in a home environment, enabling the user to self-apply a medical substance, for example insulin or hormones, without a medically trained person being present. Resulting from that, easy and safe handling of such devices is a key design aspect. Thus, depending on the intended use and type of medicament, medicament delivery devices having a varying degree of automatic functions have been developed to facilitate injection of medicaments in a reliable and safe manner.

Auto-injectors with a penetration function usually comprise a housing, a container holder carrying a medicament container with a needle and a plunger rod as well as a compression spring providing a force to move the plunger rod with respect to the housing and/or container holder. Such auto-injectors often also comprise a needle cover and an end cap protecting the tip of the needle in order to prevent injuries and contamination of the needle, when the injector is not used and/or after the injection is completed. Moreover, existing auto-injectors usually comprise an activation button that enables the user to determine the time of injection.

When said auto-injectors are actuated, the container holder and the plunger rod, which are slidable in the housing, are jointly driven towards the user's skin by the force of the compression spring, which causes the needle to penetrate the user's skin. After having reached a predetermined position, the plunger rod is moved towards the user's skin, thereby acting on a stopper inside the medicament container such that the medicament is expelled through the needle.

Some auto-injectors may further have an automatic safety function for preventing accidental needle sticks by covering the needle with the needle cover when the injector is withdrawn from the user's skin.

Prior to the actuation, the auto-injector is in a secured state, in which the end cap is attached to the housing and a movement of the plunger rod is inhibited. Initiating an injection usually requires the user to activate the injector by consecutively performing three steps. First, the user needs to remove the end cap from the housing. After that, the user needs to press the needle cover against the intended injection site with a predetermined force. Third, the user needs to press the activation button which initiates the actuation.

Such a the three-step activation as used in conventional auto-injectors and provides sufficient safety when it comes to avoiding injuries and premature medicament discharge, for example when the injector is dropped to the floor. However, from a usability point of view, an activation requiring three user interactions is disadvantageous because it increases the risk of operating errors. In the worst case such errors might block the injector, so the intended injection cannot be performed.

One of the most common operating errors is that the activation steps are not performed in the correct order, for example the activation button is pressed before the needle cover is pressed against the user's skin. Despite that, even when performed in the correct order, pressing the activation button still might be impossible, for example when the force with which the needle cover is pressed against the user's skin is less than the predetermined threshold. In this scenario, the injection cannot be initiated, either.

It is an object of the present invention to simplify the activation process of a medicament delivery device. especially a disposable single-dose auto-injector, while still preventing injuries or accidental medicament discharge.

### SUMMARY OF THE INVENTION

The above-mentioned objects are achieved, and the problems are overcome by the activation member according to independent claim 1, the subassembly according to independent claim 11 and the medicament delivery device according to independent claim 14.

Further aspects, improvements and variations are disclosed in the dependent claims, the figures and the description.

In the present application, when the term “distal” is used, this refers to the direction pointing away from the injection site, i.e. the point at which the needle penetrates the user's body in order to inject the medicament. When the term “distal part/end” is used, this refers to the part/end of the delivery device, or the parts/ends of the members thereof, which is/are located furthest away from the injection site. Correspondingly, when the term “proximal” is used, this refers to the direction pointing to the injection site. When the term “proximal part/end” is used, this refers to the part/end of the delivery device, or the parts/ends of the members thereof, which is/are located closest to the injection site.

The activation member for a medicament delivery device, according to an embodiment of the present disclosure extends along a longitudinal axis from a proximal to a distal end of the medicament delivery device. The activation member comprises an outer part and an inner part coaxially connected to the outer part. The outer part comprises a needle cover contact surface configured to contact a needle cover of the medicament delivery device. The inner part comprises at least one flexible arm extending distally and having an inclined end surface and a plunger rod support at its distal end. The inclined end surface is radially inwardly inclined in the proximal direction. The activation member further comprises at least two spring supports each extending distally and being configured to support a distal end of a drive spring of the medicament delivery device. The plunger rod support is configured to engage with a plunger rod of the medicament delivery device in order to inhibit a proximal movement of the plunger rod. The inclined end surface is configured to interact with a distal end part of the medicament delivery device, preferably a distal end cap, causing a radial outward deflection of the at least one flexible arm thereby disengaging the plunger rod support from the plunger rod, when the activation member is distally displaced by the needle cover of the medicament delivery device.

Preferably, the proximal movement of the plunger rod is inhibited by the plunger rod support when the medicament delivery device is in a non-activated state, i.e. a state prior to the distal displacement of the activation member by the needle cover, which causes a radial outward deflection of the at least one flexible arm.

Preferably, a circumferential surface of the inner part is formed of the at least one flexible arm, the at least two spring supports, and at least one slot between each flexible arm and each spring support. The at least one slot is configured to kinematically decouple the at least one flexible arm from each of the at least two spring supports in order to enable a radial outward deflection of the at least one flexible arm.

Preferably, the inner part and the outer part of the activation member overlap along a part of their length. Further preferably, the outer part is connected to or integral with the inner part via at least one support extending radially between at least one of the springs supports and a connection structure of the outer part.

Preferably, each plunger rod support has a radially inwards extending protrusion configured to form a stop surface for engagement with a distal portion of the plunger rod, so that the plunger rod is engaged with the plunger rod support, when the medicament delivery device is in a non-activated state.

Preferably, each spring support comprises a protrusion or step extending radially inwards.

Further preferably, the protrusion or step have a proximally facing support surface configured to support the distal end of the drive spring.

Preferably, the support is located at the spring support radially opposite to the protrusion or step.

Preferably, when there are two spring supports, the two spring supports are positioned with an offset between 90° and 270° in a circumferential direction of the activation member. More preferably, the two spring supports arms are positioned with an offset of 180° in a circumferential direction of the activation member.

Preferably, there are more than two spring supports distributed evenly in a circumferential direction.

Preferably, the activation member further comprises a distal spring support surface configured to interact with a distal spring of the medicament delivery device. Preferably the distal spring support surface is formed by the support or a distal end surface of the connection structure.

Preferably, the at least one flexible arm and the at least two spring supports are alternately arranged in circumferential direction.

Preferably, the proximally facing support surface of the protrusion or step of the spring support is located more proximal compared to the distally facing stop surface of the radially inwards extending protrusion of the plunger rod support.

Preferably, the inner part comprises at least two flexible arms each extending distally and each having an inclined end surface and a plunger rod support at its distal end. More preferably, the inner part comprises at least three flexible arms each extending distally and each having an inclined end surface and a plunger rod support at its distal end.

Preferably, the at least two flexible arms and the at least two spring supports are alternately arranged in circumferential direction. More preferably, the angular distribution of the at least two flexible arms and the at least two spring supports is even in a circumferential direction.

Preferably, when there are two flexible arms, the two flexible arms are positioned with an offset between 90° and 270° in a circumferential direction of the activation member. More preferably, the two flexible arms are positioned with an offset of 180° in a circumferential direction of the activation member.

Preferably, when there are two flexible arms and two spring supports alternately arranged in a circumferential direction, the angle between one flexible arm and each of the spring supports and the angle between one spring support and each of the flexible arms is 90° in a circumferential direction.

Preferably, the inner part comprises at least three spring supports.

Preferably, the spring supports are distributed in a circumferential direction with respect to the number of flexible arms, such that tilting of the drive spring of the medicament delivery device can be prevented. More preferably, the angular distribution of the spring supports in a circumferential direction is even.

The subassembly for a medicament delivery device, according to an embodiment of the present disclosure comprises a housing and an activation member according to one or more of the foregoing paragraphs or embodiments, respectively. The activation member is coaxially arranged within the housing and distally movable in relation to the housing. The subassembly further comprises a plunger rod coaxially arranged within the activation member and releasably connected to the distal end of the activation member, preferably releasably connected to the plunger rod support of the activation member. The subassembly further comprises a drive spring coaxially arranged between the plunger rod and the inner part of the activation member, preferably between the plunger rod and the spring supports of the activation member. The distal end of the drive spring is supported by the spring supports of the activation member and the proximal end of the drive spring is supported by a proximal part of the plunger rod, for example a proximal ledge of the plunger rod. The subassembly further comprises a distal end cap connected to the distal housing and a distal spring arranged between the distal end cap and the activation member, which is configured to apply a biasing force on the activation member.

Preferably, the subassembly is a distal subassembly for a medicament delivery device, and so the housing may be named a distal housing.

Preferably, the distal end cap comprises a guide hole along the longitudinal axis of the medicament delivery device configured to axially guide the distal end of the plunger rod when the activation member is distally displaced.

Preferably, the distal end cap comprises a displacement structure configured to interact with the inclined end surface of the at least one flexible arm of the activation member in order to radially deflect the at least one flexible arm such that the plunger rod support is disengaged from the plunger rod, when the activation member is distally displaced.

Preferably, the displacement structure is rigid. Preferably, the displacement structure is radially inclined, preferably with the same angle of inclination as the inclined end surface of the at least one flexible arm.

The medicament delivery device according to an embodiment of the present disclosure comprises a distal subassembly and a proximal subassembly. The distal subassembly is a subassembly according to one or more of the foregoing paragraphs.

Preferably, the proximal subassembly comprises a proximal housing connectable to the distal housing, a rotator coaxially connected to a distal inner part of the proximal housing and movable in relation to the proximal housing, a displaceable needle cover partially arranged within the proximal housing, a medicament container holder connected through the needle cover and the rotator to the proximal housing and configured to receive a medicament container. The proximal subassembly further comprises a needle cover spring disposed between the needle cover and the medicament container holder and configured to apply a biasing force on the needle cover. The needle cover is axially movable inside at least the proximal housing such that a distal end of the needle cover contacts the contact surface of the activation member whereby the activation member is distally displaced. In other words, a distal displacement of the needle cover distally displaces the activation member due to a contact of the distal end of the needle cover to the contact surface of the activation member.

Preferably, the rotator is rotationally and/or axially movable in relation to the proximal housing.

Preferably, the distal end of the needle cover comprises at least one arm extending distally and contacting the contact surface of the activation member in order to distally displace the activation member.

Preferably, the medicament delivery device is a disposable single-dose auto-injector.

The activation member according to an embodiment of the present disclosure enables the activation of a medicament delivery device in two steps, i.e. removal of the end cap and compression of the needle cover against the injection site. This reduces the risk of operating errors and leads to higher degree of automation in the injection process. Consequently, the injection process is simplified and can be easily and safely performed by non-trained users.

By the provision of at least two flexible arms on the inner part of the activation member, a uniform distribution of forces in a circumferential direction can be achieved, which is beneficial for ensuring a correct function of the medicament delivery device.

By the provision of at least two spring supports on the inner part of the activation member, a uniform distribution of forces in a circumferential direction can be achieved, which is beneficial for preventing titling of the drive spring and ensuring a correct function of the medicament delivery device.

The subassembly according to an embodiment of the present disclosure provides similar advantages.

The medicament delivery device according to an embodiment of the present disclosure is advantageous over the known devices since it can be activated with two activation steps without compromising safety aspects, e.g. the prevention of unintended product discharge due to a drop of the device. The medicament delivery device does not require an activation button, so it has less movable components and mechanisms which might cause failure.

### BRIEF DESCRIPTION OF THE DRAWINGS

The following Figures below disclose an embodiment of the invention for illustrational purposes only. In particular, the disclosure within the Figures is not meant to limit the range of protection of the invention. The embodiment shown may be modified in many ways within the scope of the claims.

FIG. 1 shows a perspective view of an activation member according to a preferred embodiment of the invention:

FIG. 2 shows a zoomed perspective view of the distal portion of an inner part of the activation member according to a preferred embodiment of the invention:

FIG. 3A shows a partially cutaway perspective view of a subassembly according to a preferred embodiment of the invention:

FIG. 3B shows a different perspective of the subassembly of FIG. 3A;

FIG. 4A shows cross-sectional view of a distal end cap of the subassembly according to a preferred embodiment of the invention;

FIG. 4B shows cross-sectional view of the distal end cap of the subassembly in engagement with the distal end of the activation member according to a preferred embodiment of the invention:

FIG. 5 shows a perspective view of a medicament delivery device according to a preferred embodiment of the invention; and

FIG. 6 shows a perspective view of a proximal subassembly of the medicament delivery device of FIG. 5.

### DETAILED DESCRIPTION OF THE DRAWINGS

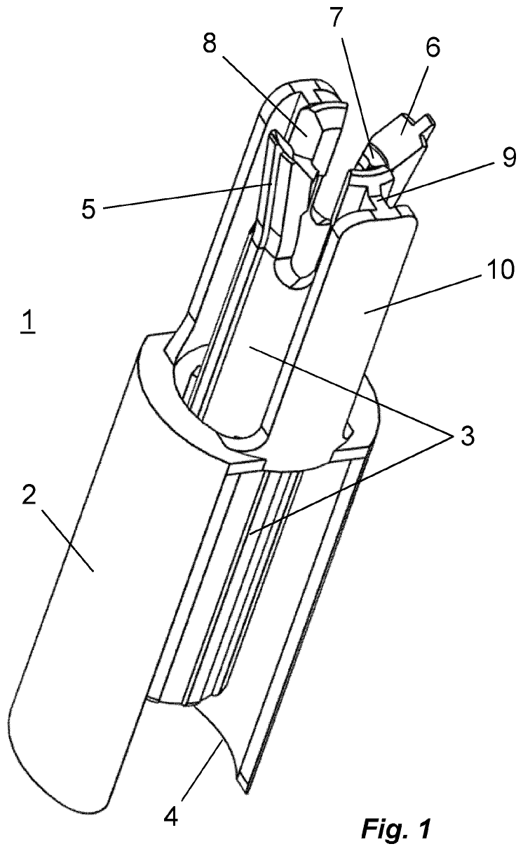

FIG. 1 shows a perspective view of an activation member 1 according to a preferred embodiment of the present disclosure. The activation member 1 comprises an outer part 2 and an inner part 3 coaxially connected to the outer part 2. The outer part 2 comprises a needle cover contact surface 4 configured to contact a needle cover of the medicament delivery device.

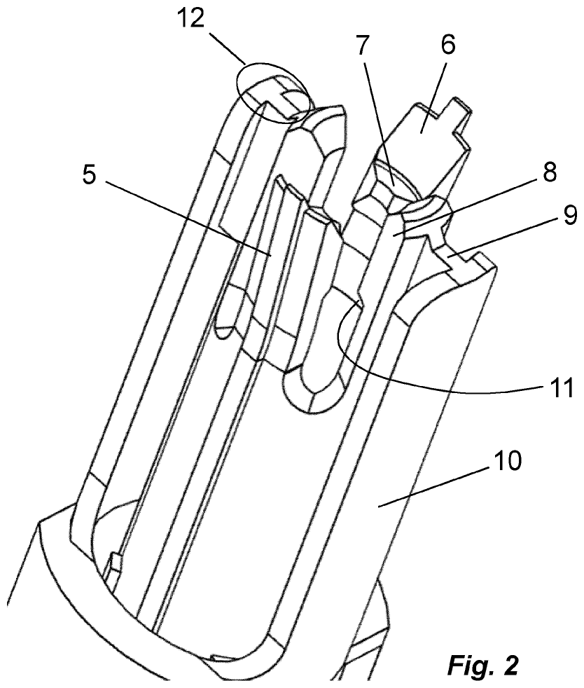

According to an embodiment, as shown in FIG. 2, the inner part 3 comprises two flexible arms 5 extending distally and having an inclined end surface 6 and a plunger rod support 7 at its distal end. The inclined end surface 6 is radially inwardly inclined in the proximal direction. As exemplarily shown, the plunger rod support 7 radially inwardly protrudes from the flexible arm 5.

According to an embodiment, the inner part 3 of the activation member 1 further comprises two spring supports 8 each extending distally. As shown, the spring supports 8 and the flexible arms 5 are distributed alternatingly along the circumference of the inner part 3, with a slot therebetween.

According to an embodiment, the outer part 2 is connected to or integral with the inner part 3 via two supports 9 extending radially between the spring supports 8 and a connection structure 10 of the outer part 2.

As further shown in FIG. 2, each spring support 8 comprises a protrusion or step extending radially inwards and having a proximally facing support surface 11 configured to support the distal end of a drive spring of the medicament delivery device. According to an embodiment. the support 9 is located radially opposite to the protrusion or step of the spring support 8. According to an embodiment, a distal spring support surface 12 is formed by the support 9 or a distal end surface of the connection structure 10.

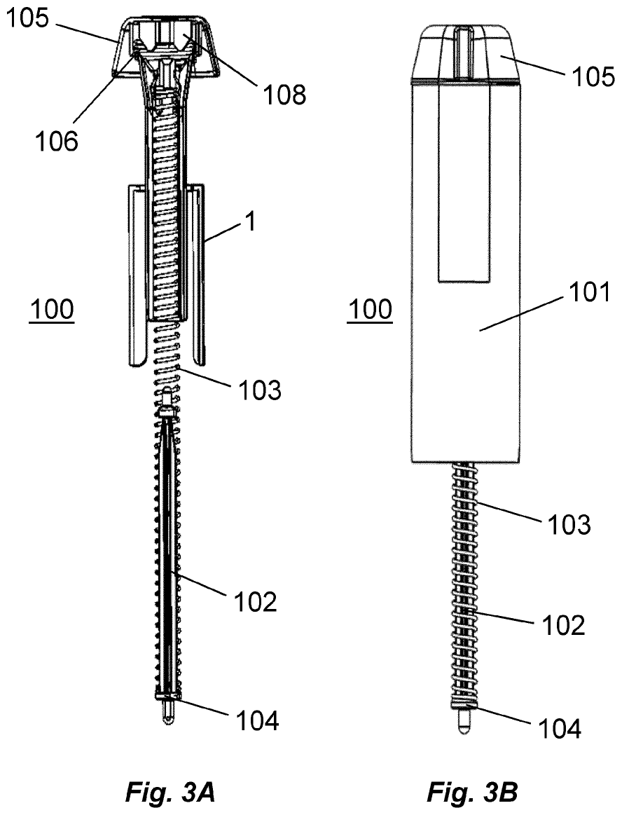

FIGS. 3A and 3B show perspective views of a subassembly 100 according to a preferred embodiment of the present disclosure. The subassembly 100 comprises a housing 101 (not shown in the cross-sectional view of FIG. 3A) and an activation member 1 (not visible in FIG. 3B) according to one of the embodiments described in the foregoing paragraphs. The activation member 1 is coaxially arranged within the housing 101. The shown subassembly 100 further comprises a plunger rod 102 coaxially arranged within the activation member 1 and releasably connected to the plunger rod support 7 of the activation member 1. The shown subassembly 100 further comprises a drive spring 103 coaxially arranged between the plunger rod 102 and the spring supports 8 of the activation member 1. which is configured to proximally displace the plunger rod 102, when the plunger rod 102 is released from its engagement with the plunger rod support 7 of the activation member 1 due to a deflection of the flexible arms 5. In FIG. 3 the drive spring 103 is shown in an expanded state and so the plunger rod 102 is actually axially outside the activation member 1. However, in an assembled state the plunger rod 102 will be coaxially arranged within the activation member 1.

The distal end of the drive spring 103 is supported by the spring supports 8 of the activation member 1 and the proximal end of the drive spring 103 is supported by a proximal part of the plunger rod 102, for example a proximal ledge 104 of the plunger rod 102. The subassembly further comprises a distal end cap 105 connected to the housing 101 and a distal spring 106 arranged between the distal end cap 105 and the activation member 1.

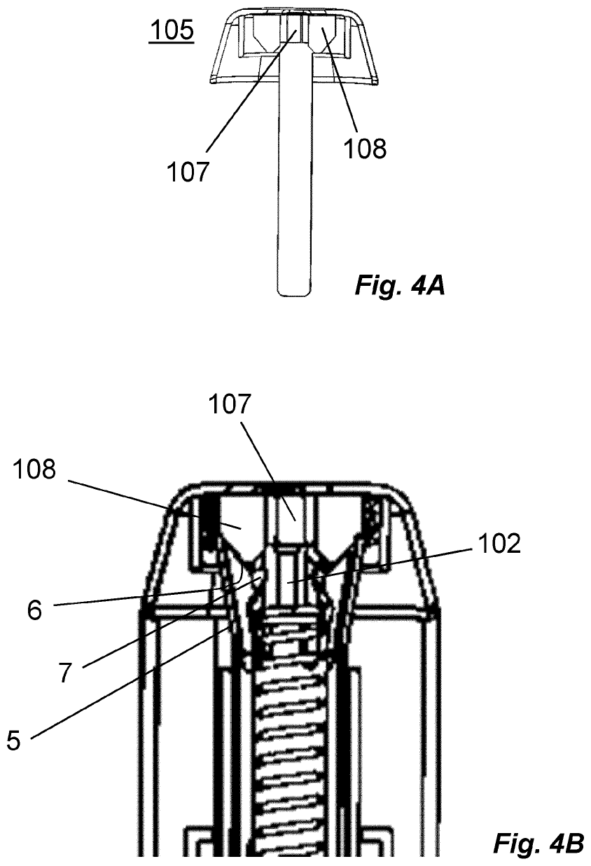

FIG. 4A shows an embodiment of the present disclosure in which the distal end cap 105 comprises a guide hole 107 and a displacement structure 108. As shown in FIG. 4B, the guide hole 107 is configured to axially guide the distal end of the plunger rod 102 when the activation member 1 is distally displaced. In this position, the displacement structure 108 interacts with the inclined end surface 6 of the flexible arms 5 of the activation member 1 and thereby radially outwardly deflects the flexible arms 5, such that the distal end of the plunger rod 102 is released from the plunger rod support 7. According to an embodiment, the displacement structure 108 is radially inclined with the same angle of inclination as the inclined end surface 6 of the flexible arms 5.

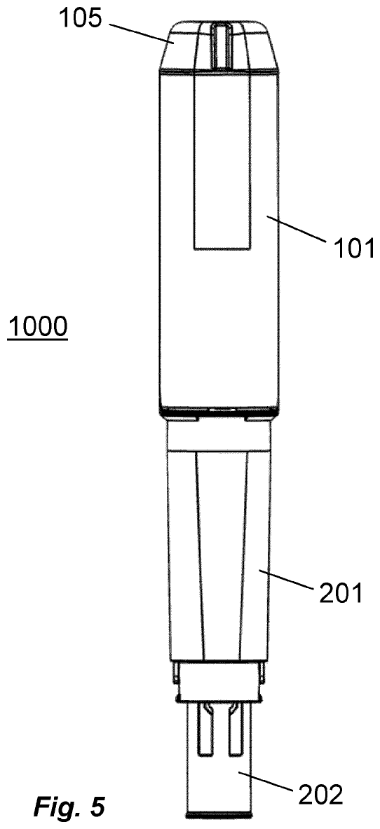

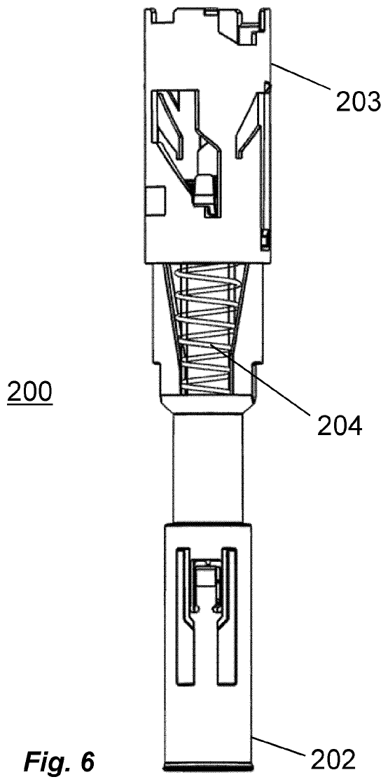

The medicament delivery device 1000 according to a preferred embodiment of the present disclosure is shown in FIG. 5 and comprises a distal subassembly 100 and a proximal subassembly 200 (see FIG. 6). The distal subassembly 100 is a subassembly 100 according to one of the embodiments described in the foregoing paragraphs.

According to an embodiment, the proximal subassembly 200 comprises a proximal housing 201 connectable to the distal housing 101 of the distal subassembly 100 and a displaceable needle cover 202 partially arranged within the proximal housing 201.

As further shown in FIG. 6, according to an embodiment, the proximal subassembly 200 further comprises a rotator 203 coaxially connected to the distal inner part of the proximal housing 201 (see FIG. 5). The rotator 203 is movable in relation to the proximal housing 201. According to an embodiment, the proximal subassembly 200 further comprises a medicament container holder (not visible in FIGS. 5 and 6) connected through the needle cover 202 and the rotator 203 to the proximal housing 201.

According to an embodiment, the proximal subassembly 200 further comprises a needle cover spring 204 disposed between the needle cover 202 and the medicament container holder and configured to apply a biasing force on the needle cover 202. The needle cover 202 is axially movable inside at least the proximal housing 201 such that the distal end of the needle cover 202 contacts the contact surface 4 of the activation member 1, when the needle cover is moved distally in order to distally displace the activation member 1.

While the invention has been illustrated and described in detail in the drawings and the foregoing description, such illustration and description are to be considered illustrative or exemplary and not restrictive. It should be noted that the invention covers all further features shown in the figures individually although they may not have been described in the afore description and it will be understood that changes and modifications may be made by those of ordinary skill within the scope of the following claims. In particular, the present invention covers further embodiments with any combination of features from different embodiments described above and below.

Furthermore, in the claims the word “comprising” does not exclude other elements or steps, and the terms “a”, “an” “first”, “second” etc. do not exclude a plurality. A single unit may fulfil the functions of several features recited in the claims. The terms “essentially”, “about”, “approximately” and the like in connection with an attribute or a value particularly also define exactly the attribute or exactly the value, respectively. Any reference signs in the claims should not be construed as limiting the scope.

1-16. (canceled)

17. An activation member for a medicament delivery device, wherein the activation member extends along a longitudinal axis from a proximal to a distal end, the activation member comprising:
an outer part and an inner part coaxially connected to the outer part, wherein the outer part comprises a needle cover contact surface configured to contact a needle cover of the medicament delivery device, and wherein the inner part comprises at least one flexible arm extending distally and having an inclined end surface and a plunger rod support at its distal end, the inclined end surface being in proximal direction inclined in a radial inward direction; and
at least two spring supports each extending distally and being configured to support a distal end of a drive spring of the medicament delivery device, wherein the plunger rod support is configured to engage with a plunger rod of the medicament delivery device in order to inhibit a proximal movement of the plunger rod, and wherein the inclined end surface is configured to interact with a distal end part of the medicament delivery device, causing a radial outward deflection of the at least one flexible arm thereby disengaging the plunger rod support from the plunger rod, when the activation member is distally displaced by the needle cover.

18. The activation member of claim 17, wherein a circumferential surface of the inner part is formed of the at least one flexible arm, the at least two spring supports, and at least one slot between each flexible arm and each spring support, wherein the at least one slot is configured to kinematically decouple the deflection of each flexible arm from the spring supports.

19. The activation member of claim 17, wherein the inner part and the outer part overlap along a part of their length.

20. The activation member of claim 17, wherein the outer part is connected to or integral with the inner part via at least one support extending radially between at least one of the spring supports and a connection structure of the outer part.

21. The activation member of claim 17, wherein each plunger rod support has a radially inwards extending protrusion configured to form a stop surface for engagement with a distal portion of the plunger rod.

22. The activation member of claim 17, wherein each spring support comprises a protrusion or step extending radially inwards and having a proximally facing support surface configured to support the distal end of the drive spring.

23. The activation member of claim 17, further comprising a distal spring support surface configured to interact with a distal spring of the medicament delivery device, wherein the distal spring support surface is formed by the support or a distal end surface of the connection structure.

24. The activation member of claim 17, wherein the at least one flexible arm and the at least two spring supports are alternately arranged in circumferential direction.

25. The activation member of any one of claim 24, wherein the proximally facing support surface of the protrusion or step of the spring support is located more proximal compared to the distally facing stop surface of the radially inwards extending protrusion of the plunger rod support.

26. A subassembly for a medicament delivery device, the subassembly comprising:
a distal housing;
an activation member according to any one of the preceding claims, wherein the activation member is coaxially arranged within the distal housing and distally movable in relation to the distal housing;
a plunger rod coaxially arranged within the activation member and releasably connected to the plunger rod support of the activation member;
a drive spring coaxially arranged between the plunger rod and the spring supports of the activation member, wherein a distal end of the drive spring is supported by the spring supports and a proximal end of the drive spring is supported by a proximal ledge of the plunger rod;
a distal end cap connected to the distal housing; and
a distal spring arranged between the distal end cap and the activation member configured to apply a biasing force on the activation member.

27. The subassembly of claim 26, wherein the distal end cap comprises a guide hole along the longitudinal axis of the medicament delivery device configured to axially guide the distal end of the plunger rod when the activation member is distally displaced.

28. The subassembly of claim 26, wherein the distal end cap comprises a displacement structure configured to interact with the inclined end surface of the at least one flexible arm in order to radially deflect the at least one flexible arm such that the plunger rod support is disengaged from the plunger rod, when the activation member is distally displaced.

29. The subassembly of claim 28, wherein the displacement structure is rigid and is radially inclined, preferably with the same angle of inclination as the inclined end surface of the at least one flexible arm.

30. A medicament delivery device comprising a distal subassembly and a proximal subassembly, wherein the distal subassembly is the subassembly of claim 26.

31. The medicament delivery device of claim 30, wherein the proximal subassembly comprises:
a proximal housing connectable to the distal housing;
a rotator coaxially connected to a distal inner part of the proximal housing and movable in relation to the proximal housing;
a displaceable needle cover partially arranged within the proximal housing;
a medicament container holder connected through the needle cover and the rotator to the proximal housing and configured to receive a medicament container; and
a needle cover spring disposed between the needle cover and the medicament container holder and configured to apply a biasing force on the needle cover,
wherein the needle cover is axially movable inside at least the proximal housing such that a distal end of the needle cover contacts the contact surface of the activation member whereby the activation member is distally displaced.

32. The medicament delivery device of claim 30, wherein the medicament delivery device is a disposable single-dose auto-injector.
